(19) United States
(12) Patent Application Publication
(10) Pub. No.: US 2026/0060841 A1
(43) Pub. Date: Mar. 5, 2026

(54) EYEDROP ASSIST DEVICE AND METHOD OF USE

(71) Applicant: Massachusetts Institute of Technology, Cambridge, MA (US)

(72) Inventors: Benjamin Lahner, Somerville, MA (US); Seung Hyeon Shim, Fremont, CA (US); Carly Smith, Orefield, PA (US); Jesse George-Akpenyi, Torrance, CA (US)

(21) Appl. No.: 19/381,128

(22) Filed: Nov. 6, 2025

Related U.S. Application Data
(63) Continuation of application No. PCT/US24/29267, filed on May 14, 2024.
(60) Provisional application No. 63/502,412, filed on May 15, 2023.

(51) Int. Cl. A61F 9/00; A61B 17/00

(52) U.S. Cl. CPC A61F 9/0026; A61B 2017/00725

(57) ABSTRACT

An eyedrop assist device is configured for use with an eyedrop bottle in an environment defining a horizontal. To that end, the eyedrop assist device has a body with a proximal end and a distal end. Importantly, the body has a receiving region to receive the eyedrop bottle and forming a longitudinal axis. The eyedrop assist device also has an angle calibrator configured to produce first indicium identifying when the longitudinal axis of the receiving region forms a first angle, and a tilt sensor. Preferably, the tilt sensor is configured to produce second indicium when the longitudinal axis of the longitudinal axis of the receiving region is tilted to form a second angle relative to the horizontal. Since they are for different purposes, the first and second angles are different angles.

### PRIORITY

This patent application is a continuation of PCT Application No. PCT/US2024/029267 entitled “EYE DROP POSITIONING DEVICE WITH HAPTIC FEEDBACK” filed May 14, 2024, which claims the benefit from provisional U.S. patent application No. 63/502,412, filed May 15, 2023, entitled, “EYE DROP POSITIONING DEVICE WITH HAPTIC FEEDBACK” and naming Benjamin Lahner, Seung Hyeon Shim, Carly Smith, and Jesse C. George-Akpenyi as inventors, the disclosures of which is incorporated herein, in its entirety, by reference.

### GOVERNMENT RIGHTS

None

### FIELD

Illustrative embodiments of the invention generally relate to eye medicine applications and, more particularly, various embodiments of the invention relate to eye drop dispensing.

### BACKGROUND

Conventional eyedrop bottles are notoriously difficult to manage for several reasons. Users must guess as to when to dispense the medication from the eyedrop bottle due to often compromised motor skills, tremors, lack of coordination, and small size of the eyedrop bottle. Undesirably, this often leads to misplaced drops and inefficient application.

Additionally, using these traditional eyedrop bottles requires users to simultaneously squeeze the bottle with two fingers, accurately aim the nozzle at a specific distance from the eye, and maintain this position while also tilting their head. This multi-step process is not only cumbersome but can be particularly problematic for elderly users or those with physical limitations, who may find it difficult to coordinate these actions smoothly.

As such, the current design of eyedrop bottles complicates what should be a simple task. It demands a level of dexterity and coordination that can be challenging for many users. This often results in frustration and ineffective medication administration.

### SUMMARY OF VARIOUS EMBODIMENTS

In accordance with one embodiment of the invention, an eyedrop assist device is configured for use with an eyedrop bottle in an environment defining a horizontal. To that end, the eyedrop assist device has a body with a proximal end and a distal end. Importantly, the body has a receiving region to receive the eyedrop bottle and forming a longitudinal axis. The eyedrop assist device also has an angle calibrator configured to produce first indicium identifying when a first angle of head tilt of a user is calibrated with the longitudinal axis of the receiving region. A tilt sensor is configured to produce second indicium when the longitudinal axis of the receiving region is tilted to form a second angle, relative to the horizontal. Since they are for different purposes, the first and second angles are different angles.

The angle calibrator may include a proximal aperture and a distal aperture. As such, alignment of the proximal and distal apertures relative to the head tilt of the user may be the first indicium. In this example, the first indicium includes light visible through the proximal and distal apertures. The tilt sensor may include a ball inclinometer. For example, to notify the person using the eyedrop assist device of an appropriate amount of tilt, the second indicium may include haptic or audio feedback.

The eyedrop assist device also may have an eyepiece at the proximal end of the body and formed from an eyepiece material. Preferably, the eyepiece material is more pliable than the body material. Moreover, those skilled in the art may select appropriate first and second angles. For example, the first angle may be between about −5 and +5 degrees relative to the horizontal, while the second angle may be between about 15 degrees and 35 degrees relative to the horizontal.

To facilitate use, the receiving region is configured to expose a portion of the eyedrop bottle when the eyedrop bottle is positioned within the receiving region. This enables a person to directly apply pressure to the bottle. In some embodiments, the eyedrop assist device also includes the eyedrop bottle. To secure the eyedrop bottle in the eyedrop assist device, the receiving region may include a retainer configured to substantially unmovably (without force) secure the eyedrop bottle within the receiving region.

In accordance with another embodiment, a method for a person to apply eyedrops to the user's eye secures an eyedrop bottle in an eyedrop assist device, and then abuts the eyedrop assist device with the eyedrop bottle against the person's face so that a proximal portion of the eyedrop assist device at least partially circumscribes the person's eye orbit. Next, the method orients (while abutting) the eyedrop assist device relative to the person's face to a prescribed orientation using a first indicium produced by an angle calibrator. After orienting, the method tilts the user's head (e.g., the user tilts under their own power) while maintaining the eyedrop assist device substantially in the prescribed calibration orientation. Upon receipt of a second indicium from the tilt sensor after tilting, the user stops the tilting their head. The user then may apply pressure to the eyedrop bottle after receiving the second indicium to dispense a medication liquid from the eyedrop bottle.

In accordance with other embodiments, an eyedrop assist device includes a monocular configured to receive an eyedrop container and provide a predetermined spatial relationship between a dispensing end of the eyedrop container and an eye of the user. In addition, the eyedrop assist device also has an eyepiece configured to be affixed to a proximal end of the monocular and provide a resilient contact point between the eyedrop assist device and an eye orbit that surrounds the eye. In preferred embodiments, the eyedrop assist device is configured to provide feedback to the user in response to the eyedrop assist device being aligned with a line of sight of the eye and when tilted to a predetermined angle.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1A schematically shows a user holding an eyedrop assist device in a level orientation in accordance with illustrative embodiments of the invention.

FIG. 1B schematically shows the user holding the eyedrop assist device in an eyedrop dispensing orientation in accordance with illustrative embodiments of the invention.

FIG. 2A schematically shows an eyedrop self-administration technique in accordance with illustrative embodiments of the conventional art.

FIG. 2B schematically shows an eyedrop location based on a neutral neck extension in accordance with illustrative embodiments of the invention.

FIG. 2C schematically shows an eyedrop location based on a minimal neck extension in accordance with illustrative embodiments of the invention.

FIG. 2D schematically shows an eyedrop location based on a maximum neck extension in accordance with illustrative embodiments of the invention.

FIG. 2E schematically shows pinhole alignment in an eyedrop assist device in accordance with illustrative embodiments of the invention.

FIG. 2F schematically shows a successful eyedrop delivery to a center of a user's eye in accordance with illustrative embodiments of the invention.

FIG. 2G schematically shows an unsuccessful eyedrop delivery below a user's eye in accordance with illustrative embodiments of the invention.

FIG. 3A schematically shows a side view of an assembled eyedrop assist device with an eyedrop bottle in accordance with illustrative embodiments of the invention.

FIG. 3B schematically shows a proximal view of the assembled eyedrop assist device in accordance with illustrative embodiments of the invention.

FIG. 4 schematically shows an exploded view of the eyedrop assist device in accordance with illustrative embodiments of the invention.

FIG. 5A schematically shows views of a body of the eyedrop assist device in accordance with illustrative embodiments of the invention.

FIG. 5B schematically shows views of an end cap of the eyedrop assist device in accordance with illustrative embodiments of the invention.

FIG. 5C schematically shows views of an eyepiece of the eyedrop assist device in accordance with illustrative embodiments of the invention.

FIG. 5D schematically shows an example of an eyedrop bottle in accordance with illustrative embodiments of the invention.

FIG. 6 shows a flowchart of an eyedrop assist device assembly process in accordance with illustrative embodiments of the invention.

FIG. 7 shows a flowchart of an eyedrop dispensing process in accordance with illustrative embodiments of the invention.

### DESCRIPTION OF ILLUSTRATIVE EMBODIMENTS

Illustrative embodiments enhance the precision and ease of administering eyedrops. To that end, the eyedrop assist device has a receiving region for receiving an eyedrop bottle, as well as a pair of sensors that help the person using the eyedrop assist device to orient and position the eyedrop bottle in a beneficial position/orientation for successfully dispensing the eyedrops. Among other things, the sensors may include an angle calibrator to orient the eyedrop assist device orthogonally relative to the person's face, and a tilt sensor to ensure the appropriate angle for the person to tilt their head to facilitate successful eyedrop delivery. Details of illustrative embodiments are discussed below.

The eyedrop assist device allows predictable and precise eyedrop administration for users by (1) optimally positioning the bottle tip to reduce neck extension, (2) precisely aligning the eyedrop assist device to a pupil of an eye, (3) stabilizing the eyedrop assist device around the eye orbit to constrain unwanted motion, and (4) providing feedback to the user when they are in an appropriate position for successful eyedrop administration.

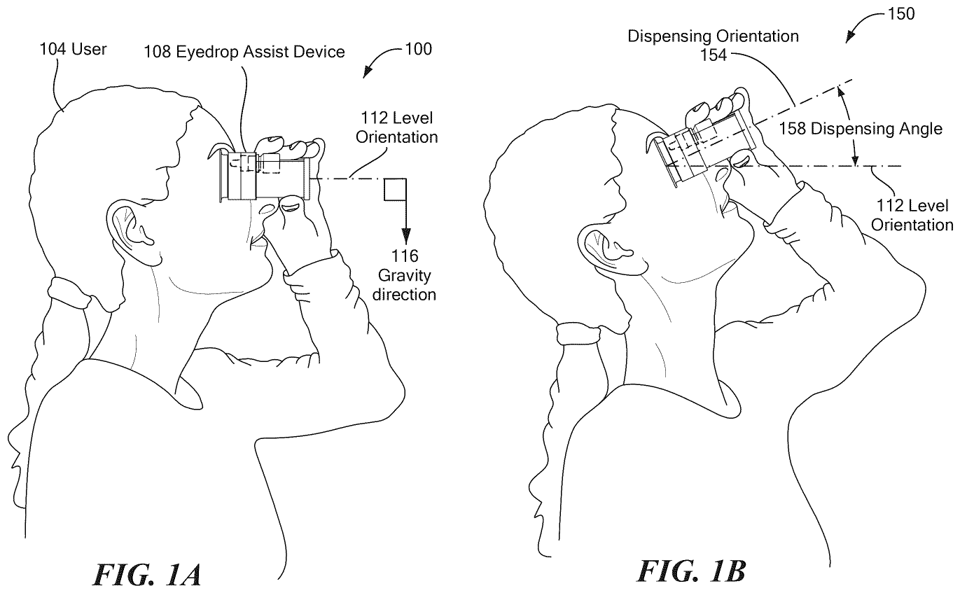

FIG. 1A schematically shows a user holding an eyedrop assist device in a level orientation 100 in accordance with illustrative embodiments of the invention. An eyedrop assist device 108 is used to provide eyedrops to an eye of the user 104. As used herein, ‘level’ means generally orthogonal to a direction of gravitational force 116. For example, if the user 104 is standing on level ground, a level orientation 112 is considered to be straight ahead with no neck tilt in any direction. If the user 104 is standing on ground that slopes downward ahead of the user 104, a level orientation 112 is considered to be looking upward compared to the plane of the ground. If the user 104 is standing on ground that slopes upward ahead of the user 104, a level orientation 112 is considered to be looking downward compared to the plane of the ground. In all cases, the eyedrop assist device 108 is in a level orientation 112 when the direction of gravity 116 is preferably orthogonally downward compared to a user's line of sight through the eyedrop assist device 108.

FIG. 1B schematically shows a user holding an eyedrop assist device 108 in an eyedrop dispensing orientation 150 in accordance with illustrative embodiments of the invention. The user 104 continues to grasp the eyedrop assist device 108 while maintaining a line of sight through the eyedrop assist device 108. The line of sight is maintained when the user detects visible light through a pair of pinholes within the eyedrop assist device 108, as discussed herein. From the level orientation 112 shown in FIG. 1A, the user 104 tilts their head backward slowly until a dispensing orientation 154 is reached. At the dispensing orientation 154, there is a dispensing angle 158 between the level orientation 112 and the dispensing orientation 154. The eyedrop assist device 108 provides feedback to the user 104 when the dispensing orientation 154 has been achieved. At this point, the user 104 may administer one or more eyedrops to the eye accurately and reliably. Broadly speaking, the eyedrop assist device 108 will function as intended as long as the user 104 detects visible light through the pair of pinholes while tilting their head at less than the dispensing angle 158 and increasing their head tilt until the dispensing orientation 154 has been reached.

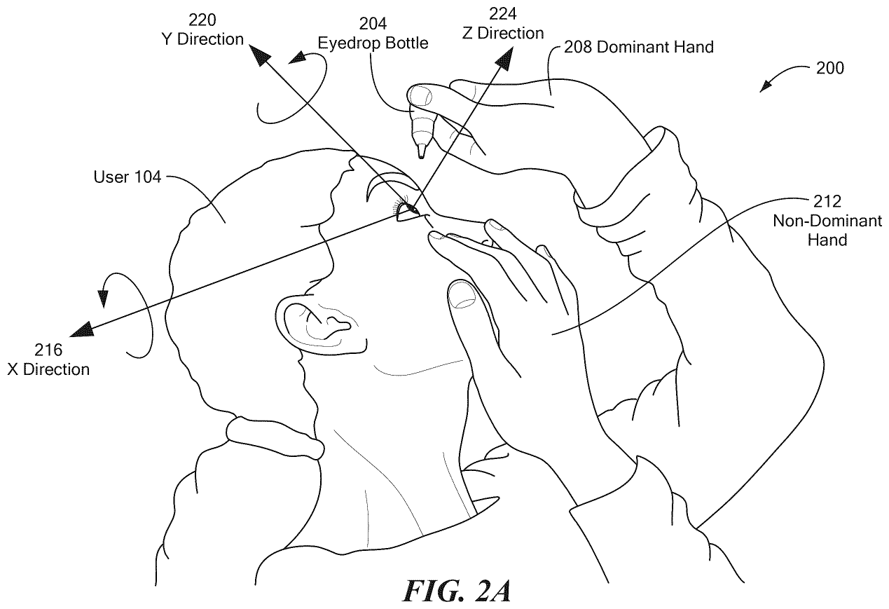

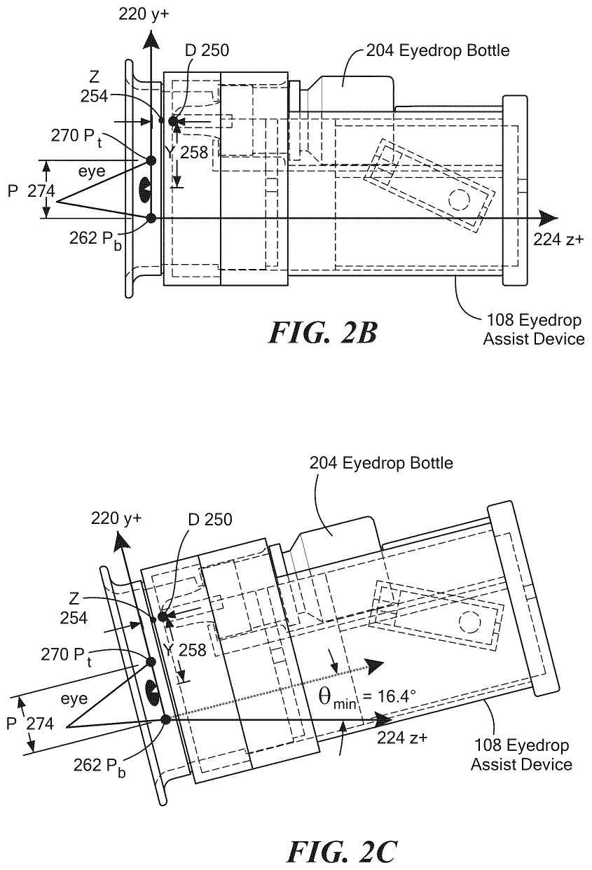

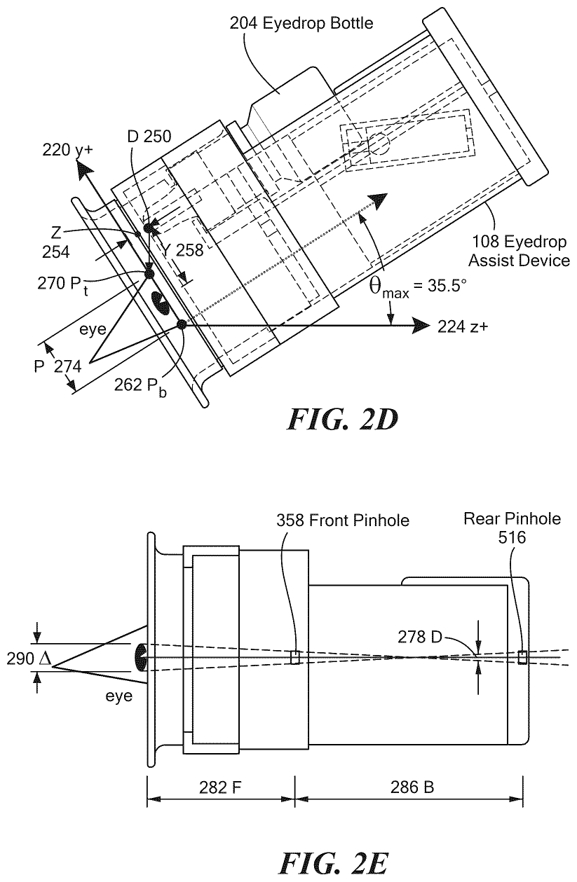

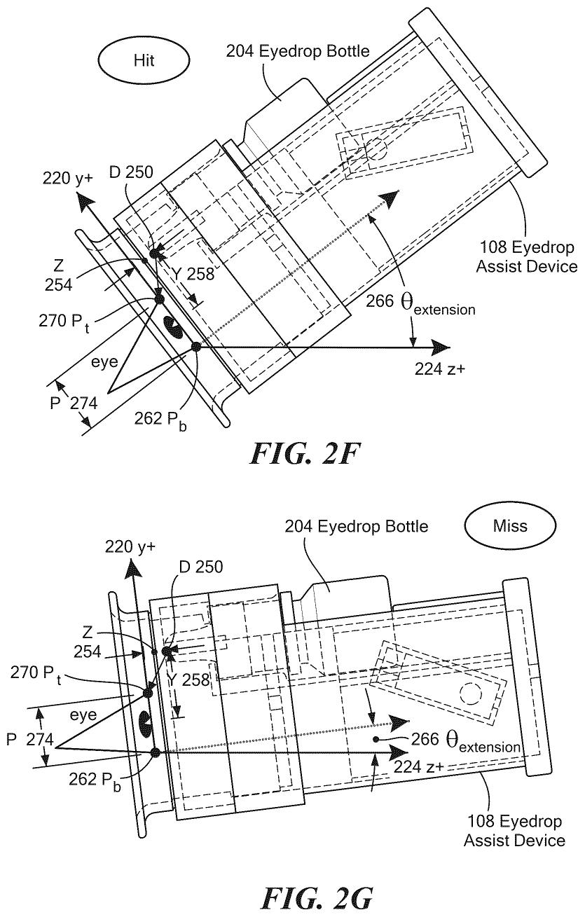

FIG. 2A schematically shows an eyedrop self-administration technique 200 in accordance with illustrative embodiments of the conventional art. Eyedrop self-administration is traditionally performed by the user 104 tilting their head back (referred to herein as neck extension), exposing the conjunctival sac of the eye by pulling down the lower eyelid with the non-dominant hand 212, positioning the eyedrop bottle 204 above the eye with the dominant hand 208, looking at the end of the eyedrop bottle 204 with the eye, and squeezing a drop of medication from the eyedrop bottle 204 into the eye. Unfortunately, this method of eyedrop application requires fine motor skills to stabilize, aim, and pinch the eyedrop bottle 204 while coordinating with the dominant 208 and non-dominant hands 212. As a result, the conventional eyedrop administration process may be unreliable and unrepeatable with many users 104.

A reference coordinate system with respect to the center of the eye surface is defined with the positive Z direction 224 moving directly away from the center of the eye, the positive Y direction 220 superior to the eye (i.e., up-down), and the positive X direction 216 moving laterally across the eye (i.e., left-right). Unconstrained translation along the X, Y, and Z axes and rotation about the X and Y axes significantly contribute to error in eyedrop self-administration. (Note that rotation about the Z axis 224 does not change the position of the bottle tip with respect to the eye).

FIG. 2B schematically shows an eyedrop location based on a neutral neck extension in accordance with illustrative embodiments of the invention. Referring to the coordinate system described in FIG. 2A, FIG. 2B shows an eyedrop D 250 positioned some distance Z 254 away from the eye (z-axis) and some distance Y 258 above the center of the eye (y-axis). Some neck extension extension 266 (see FIGS. 2C and 2D) may be required to successfully administer an eyedrop D 250 to the eye. The z-position 224 of drop D 250 must be between the z-position of the lower (P_{b}) 262 and upper (Pₜ) 270 eyelids. The distance P 274 between the lower (P_{b}) 262 and upper (Pₜ) 270 eyelids is referred to as the palpebral fissure vertical height (PFH). The eyedrop assist device 108 is assumed to be anchored to the user's face 104 by some mechanism (e.g., hand pressure of a hand holding the eyedrop assist device 108) such that the eyedrop-to-eye interface move together.

FIG. 2C schematically shows an eyedrop location based on a minimal neck extension in accordance with illustrative embodiments of the invention. A minimal neck extension θₘᵢₙ is achieved when the eyedrop D 250 falls to just inside the lower eyelid P_{b} 262. At an optimal bottle tip position, the minimum head tilt required for a successful eyedrop 250 delivery is 16.4° based on an example of user parameters discussed herein. At a lower head tilt (i.e., less than) 16.4°, the eyedrop D 250 is predicted to hit below the lower eyelid P_{b} 262, resulting in a miss.

FIG. 2D schematically shows an eyedrop location based on a maximum neck extension in accordance with illustrative embodiments of the invention. A maximum neck extension θₘₐₓ is achieved when the eyedrop D 250 falls to just inside the upper eyelid upper Pₜ 270. At an optimal bottle tip position, the maximum head tilt required for a successful eyedrop 250 delivery is 35.5°. At a higher head tilt (i.e., more than) 35.5°, the eyedrop D 250 is predicted to hit above the upper eyelid Pₜ 270, resulting in a miss.

FIG. 2E schematically shows pinhole alignment in an eyedrop assist device 108 in accordance with illustrative embodiments of the invention. Ambient light (horizontal dashed lines) that passes through the two pinholes 358, 516 of a diameter D 278 a distance B 286 apart and a distance F 282 between the pupil and left-most pinhole (i.e., proximal or front pinhole 358, compared to the user 104) will result in an illuminated region of diameter 4 (delta) 290 on the eye. It follows that if the user 104 of the eyedrop assist device 108 can see light, they have centered the eyedrop assist device 108 to within this region.

FIG. 2F schematically shows a successful eyedrop delivery to a center of a user's eye in accordance with illustrative embodiments of the invention. In this case, the eyedrop D 250 is administered at some neck extension 266 that positions the eyedrop 250 between the lower (P_{b}) 262 and upper (Pₜ) 270 eyelids, resulting in a successful administration of the eyedrop 250 to the eye. Ideally, the eyedrop 250 is delivered to the center of the user's eye. From FIGS. 2C-2D, it can be seen that the head tilt angle or neck extension (θₑₓₜₑₙₛᵢₒₙ) 266 is a critical parameter related to accurate delivery of eyedrops 250.

FIG. 2G schematically shows an unsuccessful eyedrop delivery below a user's eye in accordance with illustrative embodiments of the invention. In this case, the eyedrop 250 is administered at some neck extension θₑₓₜₑₙₛᵢₒₙ 266 that is inadequate to position the eyedrop 250 between the lower (P_{b}) 262 and upper (Pₜ) 270 eyelids, resulting in a missed eyedrop 250. Here, because of too small of a neck extension θₑₓₜₑₙₛᵢₒₙ 266, the eyedrop 250 falls on the user's face 104 below the eye. A similar diagram (not shown) could show a case for too large of a neck extension θₑₓₜₑₙₛᵢₒₙ 266, where the eyedrop 250 falls on the user's face 104 or head above the eye.

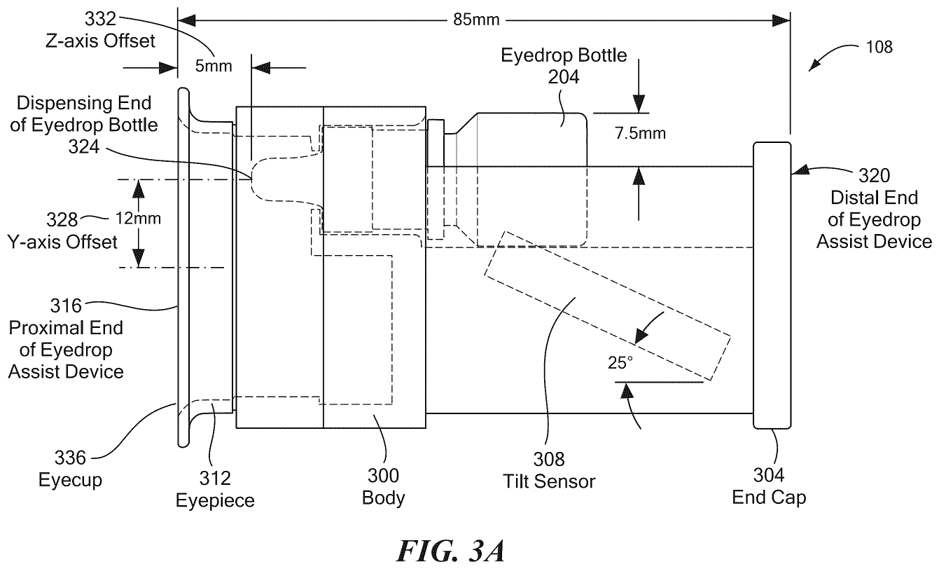

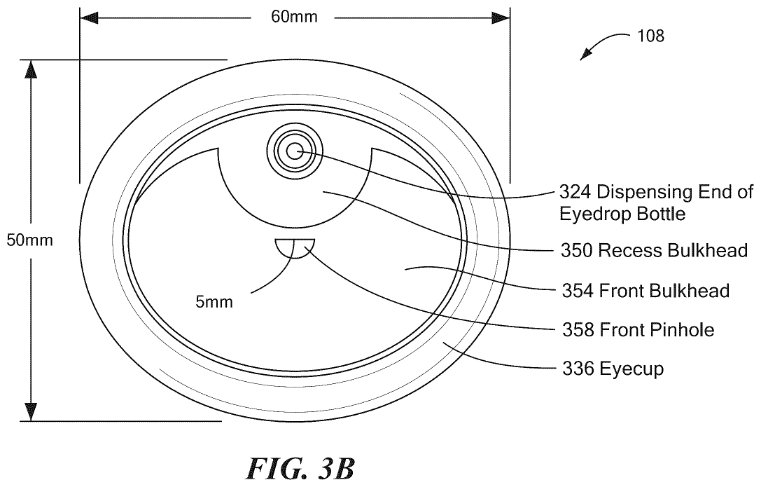

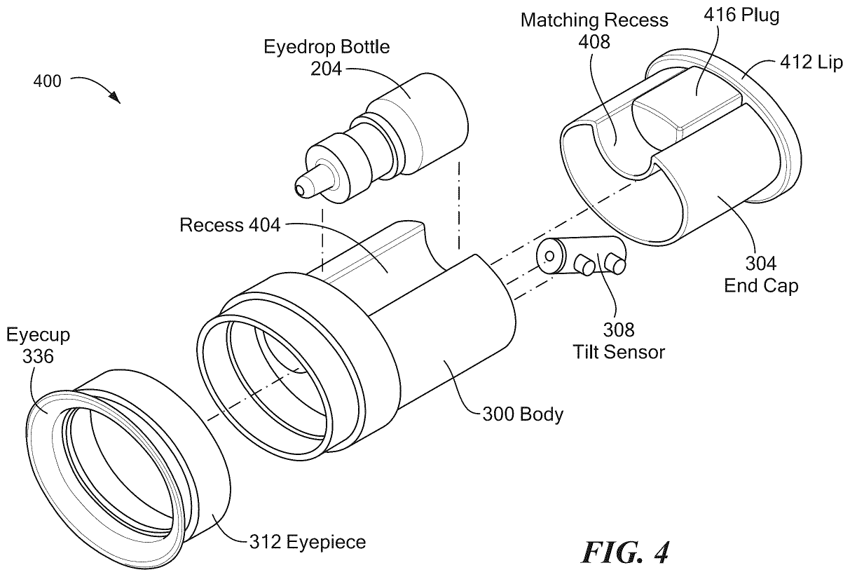

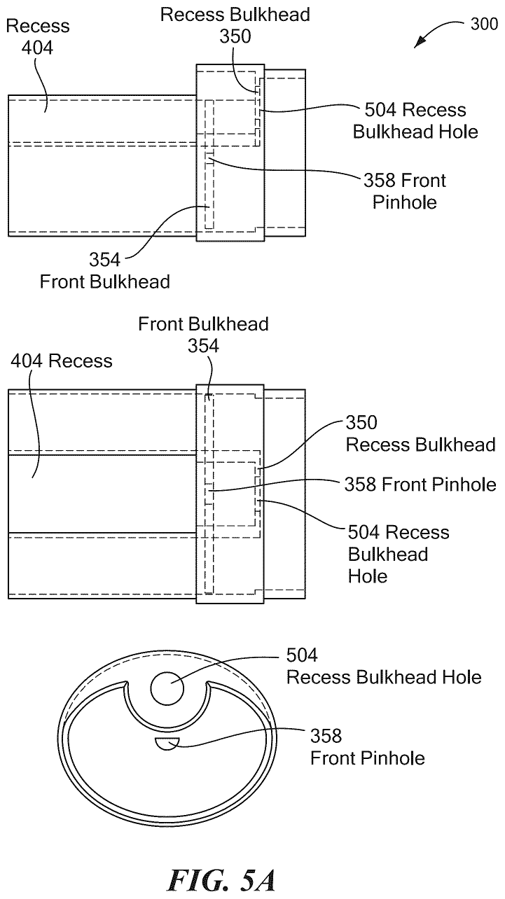

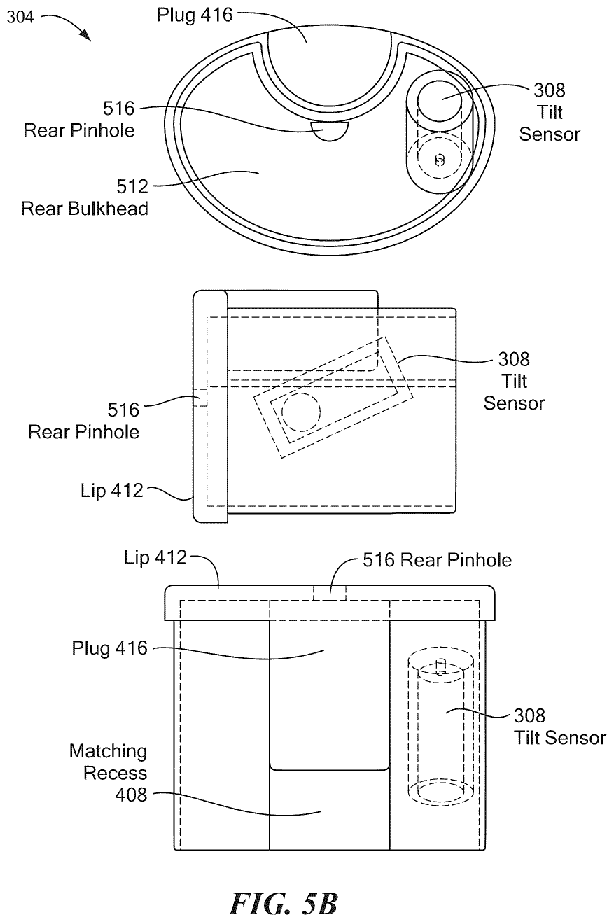

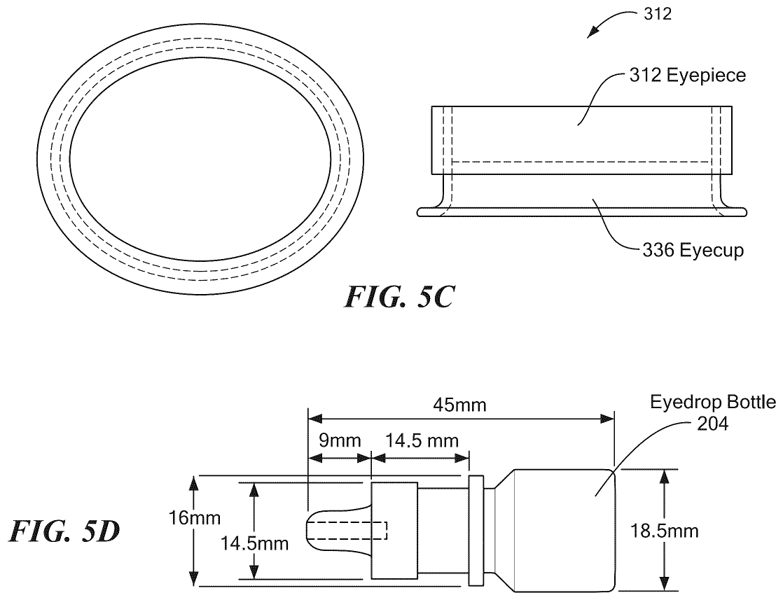

FIG. 3A schematically shows a side view of the assembled eyedrop assist device 108 with an eyedrop bottle 204 in accordance with illustrative embodiments of the invention. The assembled eyedrop assist device 108 is shown with an eyedrop bottle 204 installed, to highlight critical features and dimensions to support accurate and repeatable eyedrop 250 delivery. The eyedrop assist device 108 is configured as a familiar monocular device including a body or front portion 300, an end cap or rear portion 304, a tilt sensor 308, an eyepiece 312, and an eyecup 336. Each of the components will be discussed in more detail with respect to FIGS. 4 and 5. The proximal end 316 of the eyedrop assist device 108 that makes contact with the user's eye orbit is shown at the left side while the distal end 320 (i.e., pointing away from the user 104) is shown at the right side.

In the illustrated embodiment, the eyedrop assist device 108 may have non-critical dimensions that reflect overall utility for the intended purpose. Non-critical dimensions may include an overall length of 85 mm (millimeters) and an eyedrop bottle 204 protrusion above the body of 7.5 mm. These dimensions have been found to be optimal for a variety of differently sized users 104. However, other embodiments may have, for example, a longer overall length to accommodate very large hands of a small number of users 104.

FIG. 3A also shows three critical dimensions: an angle (25°) for the dispensing angle 158 for the tilt sensor 308, a z-axis offset for the dispensing end of the eyedrop bottle (5 mm) 324, and a y-axis offset from the centerline of the eyedrop assist device (12 mm) 328. The 25° angle reflects an optimal amount of head tilt angle or neck extension (θₑₓₜₑₙₛᵢₒₙ) 266. In other embodiments, the eyedrop assist device 108 may have different critical dimensions to meet different user needs. The critical dimensions above have been selected as being optimal for elderly users having limited neck extension (about 35°) who may receive glaucoma medication frequently. For example, different Z and Y offsets may be selected based on different user 104 needs. This may lead to a different tilt sensor 308 angle other than 25°.

The critical dimensions are required to reduce trauma and contamination risks from the dispensing end of the eyedrop bottle 324 from contacting the eye. The distance between the dispensing end of the eyedrop bottle 324 and the eye implies some level of head tilt is required to expose the surface of the eye sufficiently to deliver an eyedrop 250 successfully and reliably. Thus, the relationship between tip 324 placement and the required head tilt with equations (1), (2), and (3) is given below. The equations assume a non-curved, two dimensional (i.e., in the Y and Z planes) model of the eye with the origin set at the bottom eyelid (FIG. 2B, point P_{b} 262).

$${Θ}_{min} = \frac{π}{2} - arc tan ( \frac{Y + P / 2}{Z} ) , \qquad ( 1 )$$

where θₘᵢₙ is a minimum angle of head tilt for successful eyedrop 250 delivery, Z is a distance of the dispensing end of the eyedrop bottle 204 away from the center surface of the user's eye, Y is a distance of the dispensing end of the eyedrop bottle 204 above the center surface of the user's eye, and P is the average palpebral fissure height (the distance between the user's upper and lower eyelids).

$${Θ}_{max} = \frac{π}{2} - arc tan ( \frac{Y - P / 2}{Z} ) , \qquad ( 2 )$$

$${Θ}_{range} = {Θ}_{max} - {Θ}_{min} , \qquad ( 3 )$$

where θ_{range} is the magnitude of the range of head tilt angles for successful eyedrop 250 delivery.

10) The aim is to find the Y and Z dispensing end of the eyedrop bottle 324 placement that minimizes θₘᵢₙ and maximizes θ_{range} for a user with palpebral fissure height P. θₘₐₓ is constrained to 35°, which is the approximate maximum head tilt of the elderly population (Kuhlman, 1993; Louis and McCreary, 2021), and P=10 mm, the average palpebral fissure height in the population (Zaman et al., 1998; Roshani, 2011). Naturally, minimizing Z achieves the smallest minimum head tilt 15 (θₘᵢₙ) and greatest head tilt range (θ_{range}). A practical value for Z is 5 mm to maintain a close yet safe distance from the eye. Using these parameters, Y=12 mm (rounded to the nearest millimeter) and optimally achieves θₘᵢₙ=16.4°, θₘₐₓ=35.5°, and θ_{range}=19.1° Equations (1), (2), and (3) may be used to determine 20) a θ_{range} for different users 104 having greater or lesser amounts of neck extension 266.

The eyedrop assist device 108 precisely positions the dispensing end of the eyedrop bottle 324 5 mm away from and 12 mm above the center of the user's eye to accurately deliver eyedrops 250 when the eyedrop assist device 108 is tilted within a range between 16.4°-35.5° above the level orientation 112.

The tilt sensor 308 consists of a ball-in-tube mounted internal to the eyedrop assist device 108 at an angle of 25°. 25° is approximately in the center of the 16.6°-35.5° range, and other values close to the center of the 16.6°-35.5° range could be used. When the user's head is tilted to this 25° angle (meaning the head tilt angle or neck extension (θₑₓₜₑₙₛᵢₒₙ) is also 25°), the ball within the tilt sensor 308 rolls to an opposite end of the tube and provides a noticeable click (both audible and haptic, or felt) as an indication to the user 104 to administer an eyedrop 250. Because the tilt sensor 308 provides both audible and haptic feedback, it may be useful both for hearing-impaired users and users lacking feeling in their fingers.

Finally, the exposed side of the eyedrop bottle 204 allows the user 104 to place multiple fingers over the side of the eyedrop bottle 204 to apply pressure to distribute one or more eyedrops 250, as desired.

FIG. 3B schematically shows a proximal view of an assembled eyedrop assist device 108 in accordance with illustrative embodiments of the invention. The proximal view of the eyedrop assist device 108 includes areas of the eyedrop assist device 108 that would be viewed by the user 104 prior to placing the eyedrop assist device 108 against the eye orbit. Shown in FIG. 3B is a front bulkhead 354, a front pinhole or proximal aperture 358, a recess bulkhead 350, the eyecup 336, and the dispensing end of the eyedrop bottle 324.

The dispensing end of the eyedrop bottle 324 protrudes through a hole in the recess bulkhead 350. A front bulkhead 354 includes the front pinhole 358. The front pinhole 358 is used in conjunction with a rear pinhole 516 (see FIG. 5B) of the end cap 304 to provide an angle calibrator, as discussed herein. Referring to the coordinate system described with reference to FIG. 2A, the front 358 and rear 516 pinholes are aligned with respect to the X (left-right) and Y (up-down) directions. FIG. 3B shows common dimensions of the eyedrop assist device 108 being 60 mm wide, 50 mm in height, and having a front pinhole 358 of 5 mm and semicircular in shape.

FIG. 4 schematically shows an exploded view 400 of the eyedrop assist device 108 in accordance with illustrative embodiments of the invention. The eyedrop assist device 108 provides a monocular device to safely and reliably administer eyedrops 250 to an eye of a user 104. The eyedrop assist device 108 includes the body or front portion 300, the end cap or rear portion 304, the tilt sensor 308, and the eyepiece 312. Each of these components are described in more detail with respect to FIGS. 5A-5D. The body 300 and the end cap 304 each include alignment features that provide the angle calibrator as discussed herein.

The eyepiece 312 couples to a proximal end of the body 300, the end cap 304 couples to a distal end of the body 300, and the tilt sensor 308 is attached to either the body 300 or the end cap 304. The body 300, the end cap 304, and the tilt sensor 308 are made from rigid bio-compatible materials such as metals or polymers (e.g., ABS or PC ABS molded, extruded, and/or 3D printed plastics). Lightweight materials are preferable due to ease of holding for extended periods if necessary. The eyedrop assist device 108 may be any particular color or color combinations and textures, although easy-to-grip and retain exterior finishes are preferable. Additionally dark colors and matte finishes may be preferable to reduce light reflections and make light detection through the pinholes 358, 516 more reliable. This may also reduce ambient light within the eyedrop assist device 108 other than through the pinholes 358, 516.

The body 300 includes a longitudinal recess 404 that receives the eyedrop bottle 204. The proximal end of the recess 404 is the recess bulkhead 350 while the distal end of the recess 404 is open. The end cap 304 includes a matching recess 408 and a plug or retainer 416 that retains the eyedrop bottle 204 within the recess 404 when the end cap 304 is seated to the body 300. The end cap 304 also includes a lip or projecting feature 412 that abuts the distal end of the body 300 when seated.

FIG. 5A schematically shows views of a body 300 of the eyedrop assist device 108 in accordance with illustrative embodiments of the invention. The body (or front portion) 300 of the eyedrop assist device 108 may have a generally tubular shape and provides a main grasping surface for the person or user 104. The body 300 may be circular or oval in cross section, although an oval cross section as shown in FIG. 3B may conform more accurately to common eye orbit shapes of users 104. The body 300 has a diameter that is easy to grasp by most users 104. This reduces a need for neck extension 266 and/or wrist rotation while applying eyedrops 250, which may benefit users 104 with limited joint mobility. This also allows for single-handed eyedrop 250 delivery.

A proximal (i.e., toward the user 104) end of the body 300 may include a surface to attach the eyepiece 312, as discussed herein. A distal (i.e., away from the user 104) end of the body 300 may include one or more surfaces to attach the end cap 304 to the body 300, as discussed herein.

The body 300 may include a recess 404 to receive the eyedrop bottle 204. The recess 404 may be at least semicircular in cross section and is open along at least a portion of its length to allow a user 104 to apply finger pressure to dispense eyedrops 250 when an eyedrop bottle 204 is within the recess 404. The eyedrop bottle 204 may have a standard shape and size and/or have a range of dimensions that allow close fitment with the body 300. The recess 404 may terminate at a recess bulkhead 350 at a proximal end of the recess 404, which is located a fixed distance from the front surface of the body 300 (i.e., proximal end). The recess bulkhead 350 may have a hole 504 through which a dispensing end of the eyedrop bottle 324 protrudes when installed to the recess 404 and is pushed forward against the recess bulkhead 350.

In one embodiment, the position of the recess bulkhead 350 relative to the proximal end of the body 300 maintains a minimum distance Z 254 and Y 258 between the dispensing end of the eyedrop bottle 324 and the user's 104 eye. This may prevent the dispensing end 324 from making contact with the eye, which reduces trauma and contamination risk.

Side surfaces of the recess 404 make contact with side surfaces of the eyedrop bottle 204. This may provide a snug fit between the eyedrop bottle 204 and the recess 404, which beneficially provides resistance to finger pressure when dispensing eyedrops 250 and allows lower finger pressure to be applied than if a loose fit between the eyedrop bottle 204 and the recess 404 were provided. In one embodiment, the distal (i.e., rear) end of the recess 404 is open to slide the eyedrop bottle 204 within the recess 404 until stopped by the recess bulkhead 350.

The body 300 may include a front bulkhead 354 orthogonally oriented along a length of the recess 404. In one embodiment, the front bulkhead 354 does not extend through the recess 404 and prevents light passing directly between the ends of the body except through a front pinhole 358 in the front bulkhead 354. The front pinhole 358 is centrally located just below the recess 404 when the recess 404 is oriented along the top of the body 300 and is part of a visual alignment aid or angle calibrator discussed herein. In another embodiment, the recess bulkhead 350 may be coplanar with the front bulkhead 354—i.e., in this case a single bulkhead may extend through the cross section of the body 300 and include the recess bulkhead hole 504 through which the dispensing end of the eyedrop bottle 324 protrudes and the front pinhole 358. However, the dimensional relationships discussed herein must be observed.

In one embodiment, (if the end cap 304 does not include the tilt sensor 308) the body 300 may include the tilt sensor 308 to indicate to the user 104 when the eyedrop assist device 108 has been tilted to at least a minimum angle within an angular range. The importance of the tilt angle to successful application of eyedrops was previously discussed with reference to FIGS. 2B-2G. In one embodiment, the tilt sensor 308 is rigidly mounted to the body 300 and may include a ball inclinometer in a fixed angular orientation within the body 300. The ball inclinometer may include an object free to slide within a length of a channel, for example, a metal ball bearing within a tube. However, other shapes other than tubular may be used as long as the object slides freely under gravitational force. The tilt sensor 308 provides haptic and/or audible feedback to the user 104 when the object slides within the channel and makes contact with a hard surface at the opposite end of the channel. This occurs when the channel is oriented downward toward the opposite end of the channel and the force of gravity is greater than friction and resistive forces between the object and the channel. In one embodiment, the channel may have one or more holes in ends or sides of the channel to minimize resistive effects of air pressure to movement of the object. The one or more holes may also make object contact with ends of the tube or channel easier to hear.

FIG. 5B schematically shows views of the end cap (or rear portion) 304 of the eyedrop assist device 108 in accordance with illustrative embodiments of the invention. The end cap 304 attaches to a distal end of the body 300. In the illustrated embodiment, the end cap 304 may have a cross-section that approximates a cross-section of the body 300 and the end cap 304 may slide into the distal end of the body 300. The end cap 304 may have a lip 412 or other orthogonally projecting feature at a distal end of the end cap 304 to prevent the end cap 304 from completely sliding within the body 300. The orthogonally projecting feature or lip 412 may include a surface or plug 416 that encloses a distal end of the recess 404 of the body 300. This may retain the eyedrop bottle 204 within the recess 404 after the eyedrop bottle 204 is placed within the recess 404 and the end cap 304 is inserted into the body 300. The end cap 304 may include a lengthwise matching recess 408 that slides outside the body recess 404 as the end cap 304 is coupled to the body 300. In one embodiment, when the end cap 304 is fully seated with the body 300 and an eyedrop bottle 204 is within the recess 404, the eyedrop bottle 204 makes simultaneous contact with the recess bulkhead 350 and the rear plug 416.

The end cap 304 may include a rear bulkhead 512 orthogonally oriented along a length of the end cap 304. The rear bulkhead 512 prevents light passing directly between the ends of the end cap 304 except through a rear pinhole or distal aperture 516 in the rear bulkhead 512. In one embodiment, the rear bulkhead 512 is at the distal end 320 of the eyedrop assist device 108 and extends through the cross section of the end cap 304. In one embodiment, the rear bulkhead 512 does not extend through the rear plug 416. The rear pinhole 516 is centrally located just below the rear plug 416 when the rear plug 416 is oriented along the top of the end cap 304 and is part of the angle calibrator or visual alignment aid discussed herein.

In one embodiment, the front pinhole 358 may be a same size and shape as the rear pinhole 516. Preferably, the front pinhole 358 and the rear pinhole 516 are identically aligned within the front bulkhead 354 and the rear bulkhead 512, respectively, such that a line of sight through the two pinholes 358, 516 is parallel with side surfaces of the eyedrop assist device 108. The double pinholes precisely align the eyedrop assist device 108 to the pupil of the eye for users 104 with low visual acuity by only requiring the detection of the presence or absence of light. In another embodiment, the eyedrop assist device 108 may have more than two pinholes to accommodate larger eyedrop bottles 204. Larger eyedrop bottles 204 may potentially block light through the proximal 358 and/or distal 516 pinholes as described. For example, in addition to a proximal pinhole 358, the eyedrop assist device 108 may have two or more distal pinholes on side or alternate rear surfaces of the end cap 304.

An error in device alignment in the Y and Z dimensions is specified with Equation 4:

$$Δ = \frac{2 DF}{B} + D , \qquad ( 4 )$$

where, Δ is the error, or diameter of the projection cast on the pupil with pinholes 358, 516 aligned
D is the diameter of both pinholes
F is the distance from the pupil to the front pinhole 358
B is the distance from the front pinhole 358 to the rear pinhole 516

With the eyecup 336 anchored around their eye orbit, the user 104 can see light through the pinholes 358, 516 if the eyedrop assist device 108 is positioned within Δ. Δ 290 reflects an angle between about −5° and +5° relative to the horizontal. This pinhole alignment mechanism is conducive to the target population's vision impairments as they only need to distinguish between the presence and absence of light, not fine grained visual features such as crosshairs (Ramulu, 2009). For example, based on the user description herein, practical dimensions of 278 D=5 mm, 282 F=27 mm, and 286 B=53 mm achieve Δ 290=10.1 mm, which corresponds to a reasonable device size. For other users 104, equation (4) may be used to obtain different Δ 290 values.

In one embodiment the body 300 or the end cap 304 may include a detent (not shown) that engages a matching hole in the (other of) the body 300 and the end cap 304 when the end cap 304 is coupled to the body 300. This feature may retain the end cap 304 to the body 300 and prevent the end cap 304 from dropping away from the body 300 by gravitational force. Preferably, the retention force will be light and allow the end cap 304 to be separated from the body 300 by light finger pressure. In another embodiment, there may be a light friction fit between the end cap 304 and the body 300. In another embodiment, no detent and matching hole or friction fit is provided and the end cap 304 may be readily separable from the body 300.

In one embodiment, (if the body 300 does not include the tilt sensor 308) the end cap 304 may include the tilt sensor 308 previously discussed with respect to the body 300. The tilt sensor 308 indicates to the user 104 when the eyedrop assist device 108 has been tilted to at least a minimum angle within an angular range. It may be preferable to locate the tilt sensor 308 within the end cap 304 since the side walls of the end cap 304 may interfere with a tilt sensor 308 located in the body 300, thus requiring a longer eyedrop assist device 108 than if the tilt sensor 308 were installed in the end cap 304.

FIG. 5C schematically shows views of an eyepiece 312 of the eyedrop assist device 108 in accordance with illustrative embodiments of the invention. The eyepiece 312 is attached to the proximal end of the body 300, and in some embodiments may be removable to be replaced or cleaned. When the eyepiece 312 is brought toward the face of the user 312, an eyecup 336 of the eyepiece 312 makes contact with edges of an eye orbit of the user's eye. This provides a point of stability for the eyedrop assist device 108 and facilitates maintaining eyedrop assist device 108 alignment with the eye.

In one embodiment, the body 300 may have a friction fit to the eyepiece 312. In another embodiment, if the body 300 and the eyepiece 312 each have a circular cross section, the body 300 may provide threads that engage matching threads of the eyepiece 312 to secure the eyepiece 312 to the body 300. In another embodiment, one or more clips or fasteners may attach the eyepiece 312 to the body 300.

In one embodiment, the eyecup 336 may be made from a resilient or pliable material such as silicone or rubber. It is important to note that the eyepiece 312 of the present invention does not need or require a lens of any type.

FIG. 5D schematically shows an example of an eyedrop bottle 204 in accordance with illustrative embodiments of the invention. In one embodiment, the recess 404 of the body 300 is custom fit to receive Latanoprost bottles. In one embodiment, the eyedrop assist device 108 is designed to accommodate a 2.5 mL bottle of 0.005% Latanoprost eyedrop solution (manufactured by Bausch and Lomb, NY), which is 45 mm long and less than 20 mm in diameter. Latanoprost was specifically chosen for its frequent prescriptions to glaucoma patients. However, the present invention may be usable with any form of eyedrop bottle 204 and different eyedrop assist devices 108 may accommodate different sizes and configurations of eyedrop bottles 204 (e.g., contact lens eyedrop bottles, moisturizing solution eyedrop bottles, numbing solution eyedrop bottles, pupil dilation solution eyedrop bottles, and the like).

In one embodiment, when the eyedrop bottle 204 is installed in the recess 404, the side of the eyedrop bottle 204 may extend outward beyond the side surfaces of the body 300. This may beneficially allow greater leverage and range of motion of the user's finger to dispense eyedrops 250 by reducing required finger force.

Medication within the eyedrop bottle 204 is separate from the eyedrop assist device 108 and is not intended to come into contact with the eyedrop assist device 108. This allows the eyedrop assist device 108 to qualify as a class I medical device.

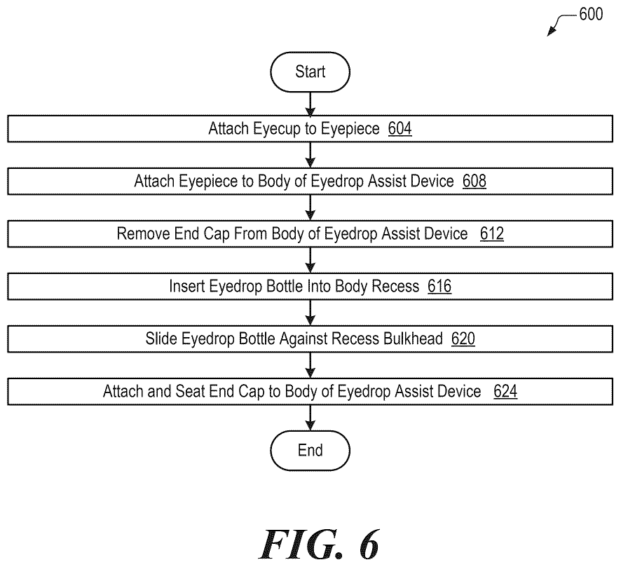

FIG. 6 shows a flowchart of an eyedrop assist device assembly process 600 in accordance with illustrative embodiments of the invention. The eyedrop assist device 108 includes various components described with respect to FIGS. 4 and 5. This process provides assembly steps for a complete eyedrop assist device 108 that is ready to use. Flow begins at block 604.

At block 604, the eyecup 336 is attached to the eyepiece 312. This step is required if the eyepiece 312 includes multiple parts but is redundant if the eyepiece 312 and eyecup 336 are a single component. In other words, a resilient eyepiece 312/eyecup 336 may be directly attached to the proximal end of the body 300. In one embodiment, an eyecup 336 made of a soft and resilient material may be attached to a more rigid eyepiece 312. Many such means of attaching the eyecup 336 to the eyepiece 312 may be utilized, including stretching edges of the eyecup 336 around a lip on an edge or within the eyepiece 312. Flow proceeds to block 608.

At block 608, The eyepiece 312 is attached to the body 300 of the eyedrop assist device 108. If contact points between the eyepiece 312 and the body 300 are rigid materials, it may be possible to use a threaded or friction attachment. Various forms of fasteners (e.g., clips, screws, etc.) may also be used. Flow proceeds to block 612.

At block 612, the end cap 304 is removed from the body 300 (if attached). The end cap 304 may be loosely attached and simply fall away from the body 300 when turned upside-down. In other embodiments, the end cap 304 and body 300 may be coupled by simple detents or friction and finger pressure may provide sufficient separation force. In another embodiment, various forms of fasteners (e.g., clips, screws, etc.) may also be used. Flow proceeds to block 616.

At block 616, an eyedrop bottle 204 containing eye medication is inserted into the body recess 404. Flow proceeds to block 620.

At block 620, the user 104 slides the eyedrop bottle 204 forward (i.e., toward the proximal end of the body 300) until the eyedrop bottle 204 makes direct contact with the recess bulkhead 350. At this point, a dispensing end of the eyedrop bottle 324 protrudes through the recess bulkhead hole 504. Flow proceeds to block 624.

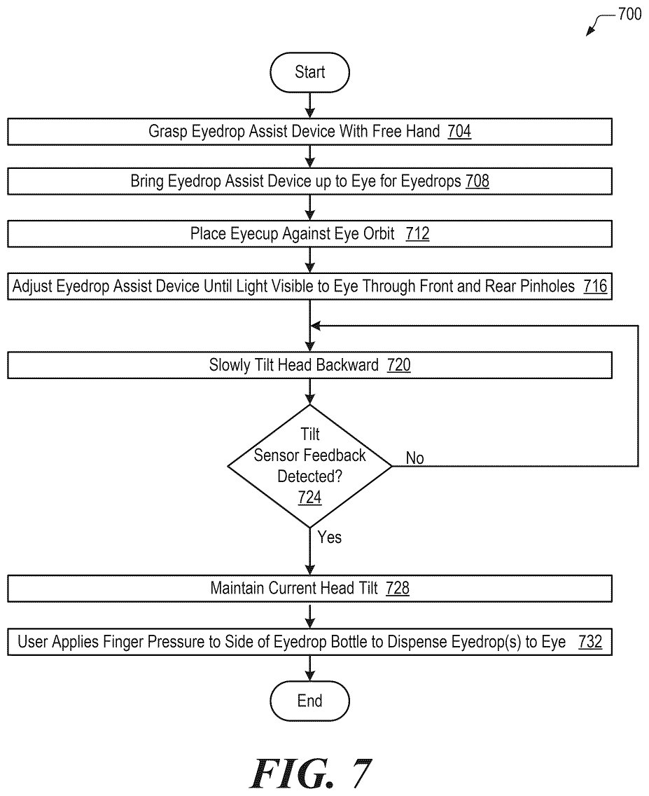

At block 624, the end cap 304 is attached and seated against the body 300 of the eyedrop assist device 108. At this point the eyedrop assist device 108 is fully assembled and ready for use, as described with respect to FIG. 7. Flow ends at block 624.

FIG. 7 shows a flowchart of an eyedrop dispensing process 700 in accordance with illustrative embodiments of the invention. The process described in FIG. 7 assumes an eyedrop bottle 204 is already installed in the eyedrop assist device 108 and is ready for use. If an eyedrop bottle 204 is not already installed, steps 612-624 of FIG. 6 should be performed prior to the steps shown in FIG. 7. Flow begins at block 704.

At block 704, a user 104 who is to receive eyedrops 250 grasps the eyedrop assist device 108 with a free hand. The eyedrop bottle 204 should be oriented along the top of the eyedrop assist device 108, when grasped. The eyedrop assist device 108 may be sized for a comfortable grasp by an adult user. Typically, a thumb of the user conforms to the bottom surface of the eyedrop assist device 108 while the other fingers conform to the top surface. It is preferable to grasp the eyedrop assist device 108 such that one or more fingertips (not the thumb) cover the side of the eyedrop bottle 204 to dispense eyedrops 250. Flow proceeds to block 708.

At block 708, the user 104 brings the eyedrop assist device 108 up to the eye that is to receive eyedrops 250. The user's head remains in the generally level orientation at this time. One of the advantages of the present invention is it eliminates the need for the hand not holding the eyedrop bottle 204 or eyedrop assist device 108 to pull down the lower eyelid, as shown in FIG. 2A. This makes the present invention much more usable for users 104 with only a single usable hand to accurately administer eyedrops. However, pulling down the lower eyelid may increase the chance of administering a successful eyedrop 250 because it increases the exposed surface area of the eye. Flow proceeds to block 712.

At block 712, the user 104 moves the eyedrop assist device 108 to place the eyecup 336 against the eye orbit. Light pressure toward the head may facilitate a stable position to anchor the eyedrop assist device 108 such that the eyedrop assist device 108 does not move or rotate. Flow proceeds to block 716.

At block 716, the user 104 adjusts the distal end of the eyedrop assist device 108 until light is visible to the eye through the front 358 and rear 516 pinholes. While maintaining light pressure to the eye orbit, the user 104 may slowly move or rotate the distal end of the eyedrop assist device 108 to align the pupil with the front 358 and rear 516 pinholes. Unless aligned, outside or ambient light will not be visible to the user 104. This step ensures the eyedrop assist device 108 is axially aligned with the eye and calibrates the head tilt of the user 104 with the angle of the tilt sensor 308, which is required for accurate eyedrop 250 application to the pupil. Flow proceeds to block 720.

At block 720, the user 104 slowly tilts their head straight backwards. This is done while maintaining the axial alignment as described in step 720. This action activates the tilt sensor 308 to provide feedback to the user 104. Flow proceeds to decision block 724.

At decision block 724, the user 104 determines if tilt sensor 308 feedback is detected. In one embodiment, the user 104 may receive audible feedback when the user tilts their head (and hence the eyedrop assist device 108) to a predetermined angle relative to horizontal (e.g., 25°, or a different angle as determined by equations (1)-(3). In another embodiment, the user 104 may receive haptic feedback when the user 104 tilts their head (and hence the eyedrop assist device 108) to a predetermined angle relative to horizontal. In one embodiment, the user 104 may receive both audible and haptic feedback when the user tilts their head (and hence the eyedrop assist device 108) to a predetermined angle relative to horizontal (e.g., 25°, etc.). When the user 104 detects tilt sensor 308 feedback, flow proceeds to block 728. As the user continues 20) to tilt their head backwards and no tilt sensor 308 feedback is detected, flow proceeds back to block 720, where the user 104 continues to tilt their head.

At block 728, the user 104 has detected tilt sensor 308 audible or haptic feedback and maintains a current head tilt angle instead of continuing to tilt their head backward. At this point, the eyedrop assist device 108 is both axially aligned to the pupil of the eye and tilted at a sufficient angle for eyedrops 250 dispensed from the eyedrop bottle 204 to drop directly on the pupil with a high degree of accuracy and repeatability. Flow proceeds to block 732.

At block 732, the user 104 applies finger pressure to a side surface of the eyedrop bottle 204 to dispense an eyedrop 250 to the eye. By successively relaxing and applying finger pressure, multiple eyedrops 250 may be dispensed. Flow ends at block 732.

The embodiments of the invention described above are intended to be merely exemplary; numerous variations and modifications will be apparent to those skilled in the art. Such variations and modifications are intended to be within the scope of the present invention as defined by any of the appended claims.

What is claimed is:

1. An eyedrop assist device for use with an eyedrop bottle in an environment defining a horizontal, the eyedrop assist device comprising:
a body having proximal end and a distal end, the body having a receiving region for removably receiving the eyedrop bottle, the receiving region having a longitudinal axis;
an angle calibrator configured to produce first indicium identifying when the longitudinal axis of the receiving region forms a first angle; and
a tilt sensor configured to produce second indicium when the longitudinal axis of the longitudinal axis of the receiving region is tilted to form a second angle relative to the horizontal,
the first and second angles being different angles.

2. The eyedrop assist device as defined by claim 1, wherein the angle calibrator comprises a proximal aperture and a distal aperture, alignment of the proximal and distal apertures producing the first indicium.

3. The eyedrop assist device as defined by claim 2, wherein the first indicium comprises light visible through the proximal and distal apertures.

4. The eyedrop assist device as defined by claim 1, wherein the tilt sensor comprises a ball inclinometer.

5. The eyedrop assist device as defined by claim 1, wherein the second indicium comprises haptic or audible feedback.

6. The eyedrop assist device as defined by claim 1, wherein the body is formed from a body material, the eyedrop assist device further comprising an eyepiece at the proximal end of the body and formed from an eyepiece material, the eyepiece material being more pliable than the body material.

7. The eyedrop assist device as defined by claim 1, wherein the first angle is between a face angle of a user and the receiving region.

8. The eyedrop assist device as defined by claim 1, wherein the second angle is between about 15 degrees and 35 degrees relative to the horizontal.

9. The eyedrop assist device as defined by claim 1, wherein the receiving region is configured to expose a portion of the eyedrop bottle when the eyedrop bottle is positioned in the receiving region.

10. The eyedrop assist device as defined by claim 1, further comprising the eyedrop bottle.

11. A method for a person to apply eyedrops to a user's eye, the method comprising:
securing an eyedrop bottle in an eyedrop assist device;
abutting the eyedrop assist device with the eyedrop bottle against the face of the user, a proximal portion of the eyedrop assist device at least partially circumscribing the user's eye;
orienting, while abutting, the eyedrop assist device relative to the user's face to a prescribed orientation using a first indicium produced by an angle calibrator;
tilting, after orienting, the user's head while maintaining the eyedrop assist device substantially in the prescribed orientation,
receiving, from a tilt sensor, a second indicium after tilting, the user stopping the tilting of the user's head upon receipt of the second indicium; and
applying pressure to the eyedrop bottle after receiving the second indicium to dispense a liquid from the eyedrop bottle.

12. The method as defined by claim 11, wherein orienting comprises aligning a proximal aperture with a distal aperture so that light is visible to the user through the distal aperture, the visible light being the first indicium.

13. The method as defined by claim 11, wherein the second indicium comprises haptic or audible feedback.

14. The method as defined by claim 11, wherein the eyedrop bottle comprises a longitudinal axis and the eyedrop assist device is oriented in an environment forming a horizontal, the first orientation comprises an angle between the eyedrop bottle longitudinal axis and a face angle of a user.

15. The method as defined by claim 11, wherein the eyedrop bottle comprises a longitudinal axis and the eyedrop assist device is oriented in an environment forming a horizontal, tilting comprising tilting the eyedrop bottle longitudinal axis to an angle of between about 15 degrees and 35 degrees relative to the horizontal.
